(19) United States
(12) Patent Application Publication
(10) Pub. No.: US 2026/0283477 A1
(43) Pub. Date: Sep. 24, 2026

(54) METHOD AND APPARATUS FOR MONITORING PERIPHERAL CIRCULATION IN HUMAN LIMBS BY INTEGRATING TEMPERATURE FEEDBACK WITH COMPOSITE OPTICAL MEASUREMENT TECHNIQUES

(71) Applicant: NATIONAL CHENG KUNG UNIVERSITY, TAINAN CITY (TW)

(72) Inventor: YI-CHUN DU, TAINAN CITY (TW)

(21) Appl. No.: 19/326,044

(22) Filed: Sep. 11, 2025

(30) Foreign Application Priority Data
Mar. 18, 2025 (TW) 114110114

(51) Int. Cl. A61B 5/02; A61B 5/00; A61B 5/01; A61B 5/0295; A61B 5/282

(52) U.S. Cl. CPC A61B 5/02007; A61B 5/01; A61B 5/0295; A61B 5/282; A61B 5/6826; A61B 5/6838; A61B 5/7235; A61B 5/7271; A61B 2560/0468; A61B 2562/0238; A61B 2562/0271

(57) ABSTRACT

A method and an apparatus for monitoring peripheral circulation in human limbs by integrating temperature feedback with composite optical measurement techniques are disclosed. The apparatus includes four measuring devices each having a temperature sensor, two electrocardiogram measuring electrodes, a light-emitting element, a reflective measuring element and a transmissive measuring element that are mounted in a clamping body. When in use, the four measuring devices clamp the extremities of a human body to detect the temperature of the extremities and automatically heat the extremities to raise the temperature, when necessary, thereby evaluating the local blood vessel stiffness and elasticity conditions of each of the extremities of the human body.

### FIELD OF THE INVENTION

The present invention relates to a method and an apparatus for monitoring peripheral circulation in human limbs, and more particularly to a method and an apparatus for monitoring peripheral circulation in human limbs by integrating temperature feedback with composite optical measurement techniques. The composite optical measurement techniques can simultaneously obtain a reflective photoplethysmography signal (rPPG) and a transmissive photoplethysmography signal (tPPG) for evaluating the local blood vessel stiffness and elasticity conditions of each of the extremities of a human body.

### BACKGROUND OF THE INVENTION

Traditionally, when evaluating vascular obstruction in the limbs, non-invasive tests such as the ankle-brachial index (ABI) are commonly used. Alternatively, invasive methods like angiography may be performed, in which a contrast agent is delivered to the examination site via a catheter to visualize the vascular pathway and observe whether any pathological changes are present.

When measuring blood pressure, the systolic and diastolic pressures of the human pulse can be measured using individual physiological characteristics of electrocardiography (ECG) and photoplethysmography (PPG).

Electrocardiography (ECG) is a method that records the heart’s electrophysiological activity in units of time through the chest. By attaching electrodes to the surface of the human skin, the electrical conduction of the heart can be detected. The results of an electrocardiogram are usually displayed as waveforms, wherein the QRS complex represents ventricular contraction.

Photoplethysmography (PPG) is a non-invasive method for detecting blood volume changes in living tissues using photoelectric means. When a light beam of a specific wavelength shines on the skin surface of the fingertip, the contraction and expansion of blood vessels with each heartbeat affect the transmission or reflection of light, while the absorption of light by the vessels also changes. These variations reflect the characteristics of blood flow.

Therefore, based on the individual physiological characteristics of PPG and ECG, for example, the peak of ECG comes from the ventricular contraction, while the peak of PPG is caused by the ventricular contraction. Therefore, the transit time of blood from the heart to the measurement site can be obtained, namely, the pulse transit time (PTT). The speed of pulse transit is directly related to blood pressure. When blood pressure is high, pulse transit is fast, and vice versa. The pulse transit time is obtained through electrocardiogram (ECG) and photoplethysmography (PPG). Combined with some conventional body parameters, the speed of the pulse transit can be obtained, and the systolic and diastolic pressures of the human pulse can be further obtained, thus realizing non-invasive and continuous blood pressure measurement.

However, when using photoplethysmography (PPG), since the light is directed onto the skin surface, if there is a temperature difference between the skin surface at the extremities and the core of the human body, the measurement may become less accurate, thereby affecting the reliability of the results.

Chinese Patent Publication No. CN110384492A discloses a control method and a wearable device for improving the accuracy of PPG heart rate measurement. The method includes: collecting a current ambient temperature and a skin temperature, and determining a reference skin temperature corresponding to the current ambient temperature based on the relationship curve between the skin temperature and the ambient temperature. If the current skin temperature is greater than or equal to the reference skin temperature, a heart rate monitoring unit is activated. If the current skin temperature is lower than the reference skin temperature, a reference PWM duty cycle at the current ambient temperature is determined according to the relationship curve between the PWM duty cycle and the ambient temperature, a heating unit is activated to heat the surrounding skin until the temperature of the surrounding skin detected by a temperature detection unit reaches the reference skin temperature, the PWM signal and the heating unit are turned off, and the heart rate monitoring unit is activated.

Although the aforementioned patent discloses controlling skin temperature to perform PPG measurement at a suitable temperature to improve the accuracy of PPG measurement and to compare the time differences in signal transmission between the extremities, it does not disclose any solution for detecting or monitoring vascular elasticity and vascular stiffness of the extremities of the human body.

### SUMMARY OF THE INVENTION

The primary object of the present invention is to provide an apparatus and a method for monitoring peripheral circulation in human limbs by integrating temperature feedback with composite optical measurement techniques. The apparatus uses temperature detection and automatic heating to capture electrocardiography (ECG) and photoplethysmography (PPG) signals at a suitable temperature at the extremities of a human body for monitoring a vascular blockage condition from the heart to the extremities of the human body. Besides, the apparatus uses composite optical measurement techniques to obtain a reflective photoplethysmography signal (rPPG) and a transmissive photoplethysmography signal (tPPG) for monitoring the local blood vessel stiffness and elasticity conditions of the extremities of the human body.

According to one aspect of the present invention, an apparatus for monitoring peripheral circulation in human limbs by integrating temperature feedback with composite optical measurement techniques is provided. The apparatus comprises four measuring devices, a heating unit, and a control unit. The measuring devices each includes a clamping body having clamping force. The clamping body is configured for clamping extremities of a human body. The clamping body has a clamping bottom surface and an opposing clamping top surface. Two electrocardiogram measuring electrodes are respectively disposed on the clamping bottom surface and the clamping top surface. A light-emitting element, a reflective measuring element and a temperature sensor are disposed on the clamping bottom surface. A transmissive measuring element is disposed on the clamping top surface. The reflective measuring element is adjacent to the light-emitting element. The transmissive measuring element faces the light-emitting element. The heating unit selectively increases a human body temperature or an ambient temperature. The control unit is connected to the heating unit and the two electrocardiogram measuring electrodes, the light-emitting element, the reflective measuring element, the transmissive measuring element and the temperature sensor of each of the four measuring devices. When in use, the four measuring devices clamp the extremities of the human body, respectively. The control unit detects the extremities of the human body through the temperature sensors of the four measuring devices to obtain an extremity temperature. When the extremity temperature of any one of the extremities of the human body falls within a predetermined range, the control unit activates the heating unit to perform heating. The control unit obtains an electrocardiogram signal through the electrocardiogram measuring electrodes of the measuring devices attached to the upper extremities. The light-emitting elements of the four measuring devices each continuously emit a light source. The control unit receives the light source through the four reflective measuring elements to obtain four reflective photoplethysmography signals and receives the light source through the four transmissive measuring elements to obtain four transmissive photoplethysmography signals. The control unit evaluates a vascular blockage condition from the heart to the extremities of the human body through peak time differences between the electrocardiogram signal and the four reflective photoplethysmography signals, or through peak time differences between the electrocardiogram signal and the four transmissive photoplethysmography signals. The control unit evaluates local blood vessel stiffness and elasticity conditions of each of the extremities of the human body through a difference between a corresponding one of the four reflective photoplethysmography signals and a corresponding one of the four transmissive photoplethysmography signals of each of the extremities of the human body.

Preferably, the predetermined range is between 31 degrees Celsius and 35 degrees Celsius.

Preferably, the electrocardiogram measuring electrode on the clamping top surface surrounds the transmissive measuring element, and the electrocardiogram measuring electrode on the clamping bottom surface surrounds the reflective measuring element and the light-emitting element.

Preferably, the reflective photoplethysmography signal has a reflective systolic time, the transmissive photoplethysmography signal has a transmissive systolic time, and the local blood vessel stiffness and elasticity conditions of each of the extremities of the human body are evaluated through a difference between the reflective systolic time and the transmissive systolic time.

Preferably, the heating unit has four heaters each mounted in the clamping body, and the heaters selectively heat the extremities of the human body directly for increasing the human body temperature.

Preferably, the heating unit is an electric heater that is arranged together with the human body in an environmental space, so that the heating unit selectively heats the environmental space for increasing the ambient temperature.

According to another aspect of the present invention, a method for monitoring peripheral circulation in human limbs by integrating temperature feedback with composite optical measurement techniques is provided. The method comprises the following steps of: providing four measuring devices to clamp extremities of a human body and a heating unit, wherein the measuring devices each include a temperature sensor, two electrocardiogram measuring electrodes, a light-emitting element, a reflective measuring element and a transmissive measuring element that are connected to a control unit, and the heating unit is connected to the control unit; detecting the extremities of the human body through the temperature sensors of the four measuring devices to obtain an extremity temperature, wherein when the extremity temperature of any one of the extremities of the human body falls within a predetermined range, the control unit activates the heating unit to perform heating; obtaining an electrocardiogram signal through the electrocardiogram measuring electrodes of the measuring devices attached to the upper extremities; emitting a light source through each of the light-emitting elements of the four measuring devices, wherein the four reflective measuring elements receive the light source to obtain four reflective photoplethysmography signals, and the four transmissive measuring elements receive the light source to obtain four transmissive photoplethysmography signals; evaluating a vascular blockage condition from the heart to the extremities of the human body through peak time differences between the electrocardiogram signal and the four reflective photoplethysmography signals or through peak time differences between the electrocardiogram signal and the four transmissive photoplethysmography signals; and evaluating local blood vessel stiffness and elasticity conditions of each of the extremities of the human body through a difference between a corresponding one of the four reflective photoplethysmography signals and a corresponding one of the four transmissive photoplethysmography signals of each of the extremities of the human body.

According to the above technical features, the present invention can achieve the following effects:
1. The temperature sensor, the electrocardiogram measuring electrodes, the light-emitting element, the reflective measuring element and the transmissive measuring element are all mounted in the clamping body, and the heating unit is selectively provided in the clamp body. When the clamping body of the measuring device clamps the extremity of the human body, the measuring device can simultaneously detect the temperature of the extremity of the human body, increase the temperature of the extremity of the human body, obtain the electrocardiogram signal, obtain the reflective photoplethysmography signal (rPPG), and obtain the transmissive photoplethysmography signal (tPPG).
2. The local blood vessel stiffness and elasticity conditions of each of the extremities of the human body can be evaluated through the difference between the reflective photoplethysmography signal and the transmissive photoplethysmography signal of each of the extremities of the human body.
3. The measuring device can simultaneously obtain two types of PPG signals from each of the extremities of the human body, including the reflective photoplethysmography signal (rPPG) and the transmissive photoplethysmography signal (tPPG). Therefore, four rPPG signals and four tPPG signals are obtained through the four measuring devices attached to the extremities of the human body, which is beneficial for computers or artificial intelligence to analyze and predict the vascular condition and the blood circulation function of human limbs.
4. The reflective photoplethysmography signal (rPPG) and the transmissive photoplethysmography signal (tPPG) can be obtained in a synchronous measurement manner or in a staged measurement manner.
5. The temperature sensor is configured for detecting the temperature of the skin at the extremities of the human body. When the temperature at the extremities of the human body is too low, the heating unit heats the extremities of the human body to increase the temperature at the extremities, thereby preventing ineffective sampling in photoplethysmography.
6. The pulse transit time difference at each of the extremities of the human body is measured based on the electrocardiogram signal and the reflective or transmissive photoplethysmography signal of each of the extremities of the human body, thereby evaluating the vascular blockage condition from the heart to the extremities of the human body.

### BRIEF DESCRIPTION OF THE DRAWINGS

FIG. 1 is a block diagram of the present invention;

FIG. 2 is an exploded view of the measuring device of the present invention;

FIG. 3 is a schematic view of the present invention when in use, wherein the four measuring devices are attached to the extremities of the human body;

FIG. 4 is a schematic view of the present invention when in use, wherein a finger is inserted and clamped in the clamping body;

FIG. 5 is a flow chart of measuring the temperature of the extremities of the human body using the apparatus of the present invention;

FIG. 6 is a flow chart of measuring photoplethysmography (PPG) according to the present invention;

FIG. 7 is a schematic diagram showing a first pulse transit time difference, a second pulse transit time difference, a third pulse transit time difference and a fourth pulse transit time difference obtained by the peak time difference between the electrocardiogram signal and the first reflective photoplethysmography signal, the second reflective photoplethysmography signal, the third reflective photoplethysmography signal and the fourth reflective photoplethysmography signal according to the present invention; and

FIG. 8 is a schematic diagram illustrating the difference between the reflective photoplethysmography signal rPPG and the transmissive photoplethysmography signal tPPG of the extremity of the human body according to the present invention.

### DETAILED DESCRIPTION OF THE PREFERRED EMBODIMENTS

Embodiments of the present invention will now be described, by way of example only, with reference to the accompanying drawings.

The present invention discloses a method and an apparatus for monitoring peripheral circulation in human limbs by integrating temperature feedback with composite optical measurement techniques.

In the present invention, the term “extremities” refers to the distal ends of the human limbs, not limited to fingers and toes, but also including the forearm, the palm, the lower leg, and the sole of the foot. In the following embodiments, the four extremities are referred to as the distal ends of the limbs, with the fingers and toes used as illustrative examples for the extremities.

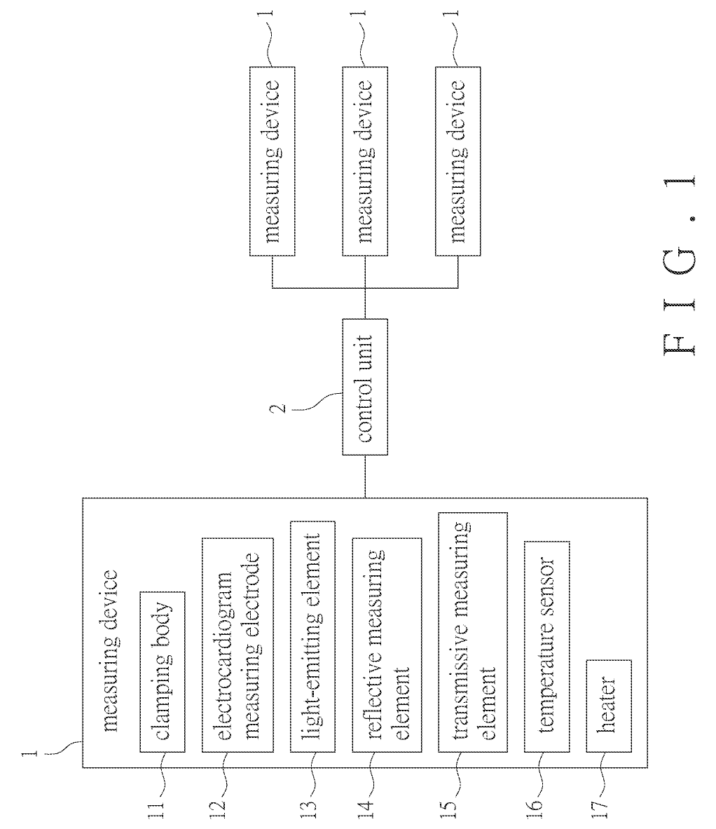

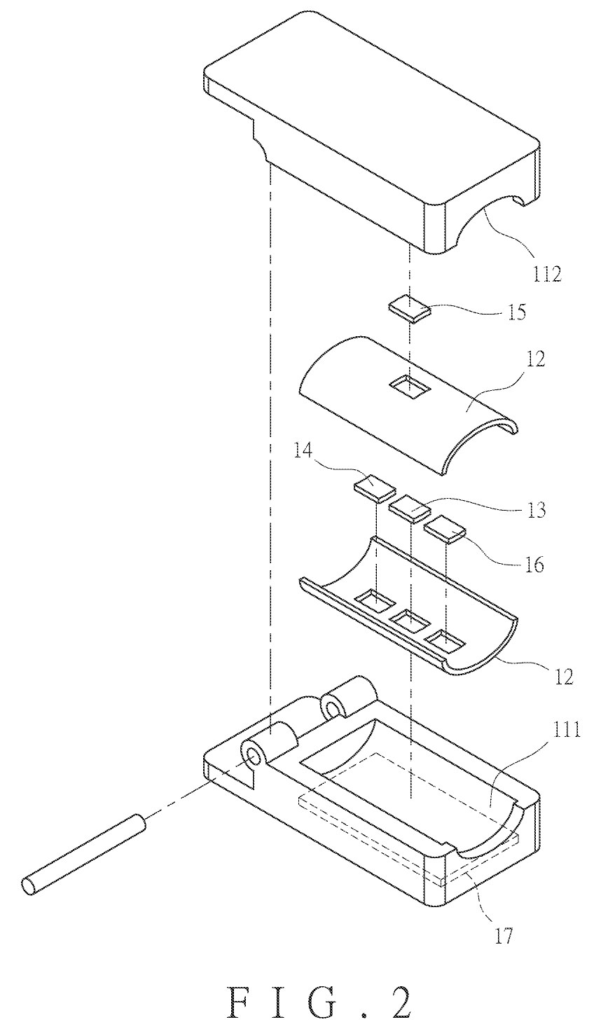

Referring to FIG. 1 and FIG. 2, the apparatus for monitoring peripheral circulation in human limbs by integrating temperature feedback with composite optical measurement techniques provided by the present invention comprises four measuring devices 1, a heating unit, and a control unit 2.

The measuring devices 1 each includes a clamping body 11 having clamping force. The clamping body 11 is configured for clamping the extremities of a human body. The clamping body 11 has a clamping bottom surface 111 and an opposing clamping top surface 112. Two electrocardiogram measuring electrodes 12 are disposed on the clamping bottom surface 111 and the clamping top surface 112, respectively. A light-emitting element 13, a reflective measuring element 14 and a temperature sensor 16 are disposed on the clamping bottom surface 111. A transmissive measuring element 15 is disposed on the clamping top surface 112. The reflective measuring element 14 is adjacent to the light-emitting element 13. The transmissive measuring element 15 faces the light-emitting element 13. Specifically, the electrocardiogram measuring electrode 12 on the clamping top surface 112 surrounds the transmissive measuring element 15. The electrocardiogram measuring electrode 12 on the clamping bottom surface 111 surrounds the reflective measuring element 14, the light-emitting element 13, and the temperature sensor 16.

The heating unit has four heaters 17 each mounted in the clamping body 11. In this embodiment, the heater is an electric heating plate. The heater 17 selectively heats the extremities of the human body directly to increase the temperature of the extremities of the human body. Specifically, the heater 17 is mounted on the clamping bottom surface 111 of the clamping body 11, so that each heater 17 can selectively heat the extremities of the human body directly to increase the temperature of the extremities of the human body.

In other embodiments, the heating unit may be an electric heater, which is arranged together with the human body in an environmental space. The heating unit heats the environmental space to increase the temperature of the extremities of the human body. In addition to the electric heater, the heating unit may be a stove, a hot air fan, a heater, an infrared heater, etc.

The control unit 2 is connected to the heating unit as well as the two electrocardiogram measuring electrodes 12, the light-emitting element 13, the reflective measuring element 14, the transmissive measuring element 15 and the temperature sensor 16 of each of the four measuring devices 1.

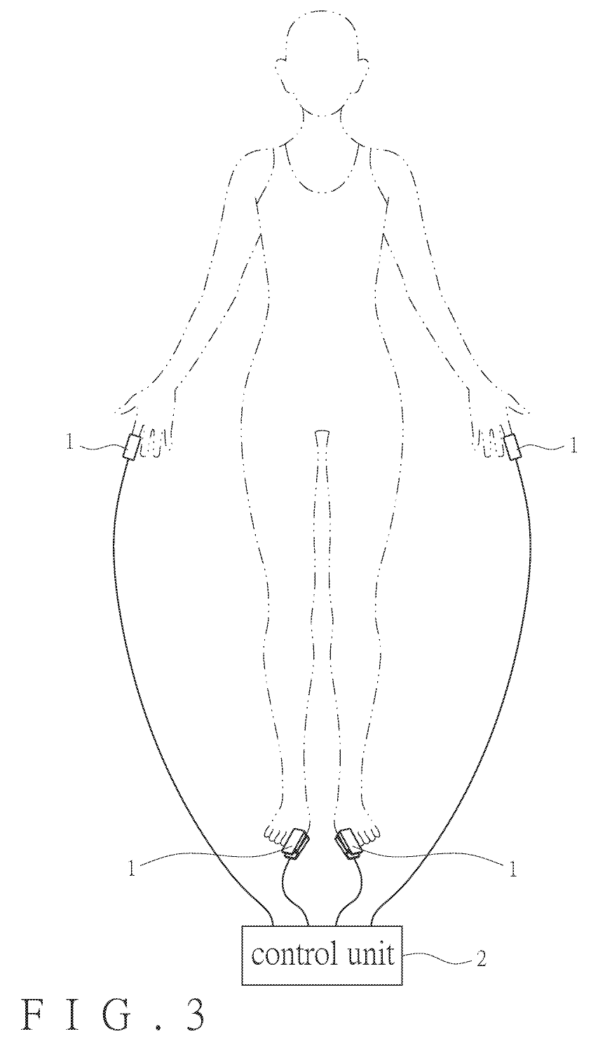

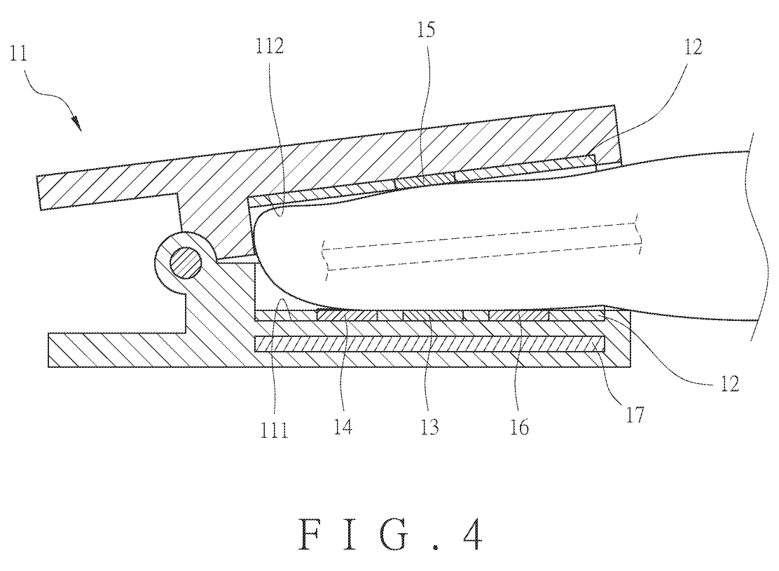

Referring to FIG. 3 and FIG. 4, when in use, the four measuring devices 1 clamp the extremities of the human body, respectively. Specifically, the four measuring devices 1 clamp the fingers of the left and right hands and the toes of the left and right feet. In this embodiment, the measuring device 1 clamps the finger as an example to describe the method for monitoring peripheral circulation in human limbs by integrating temperature feedback with composite optical measurement techniques provided by the present invention.

One of the fingers of one hand is inserted in the clamp body 11 and clamped between the clamping bottom surface 111 and the clamping top surface 112. The finger is in contact with the two electrocardiogram measuring electrodes 12, the light-emitting element 13, the reflective measuring element 14, the transmissive measuring element 15, and the temperature sensor 16.

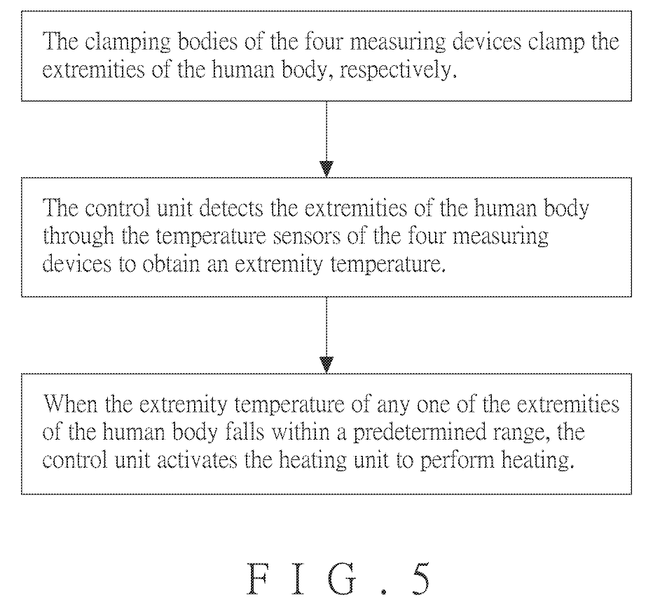

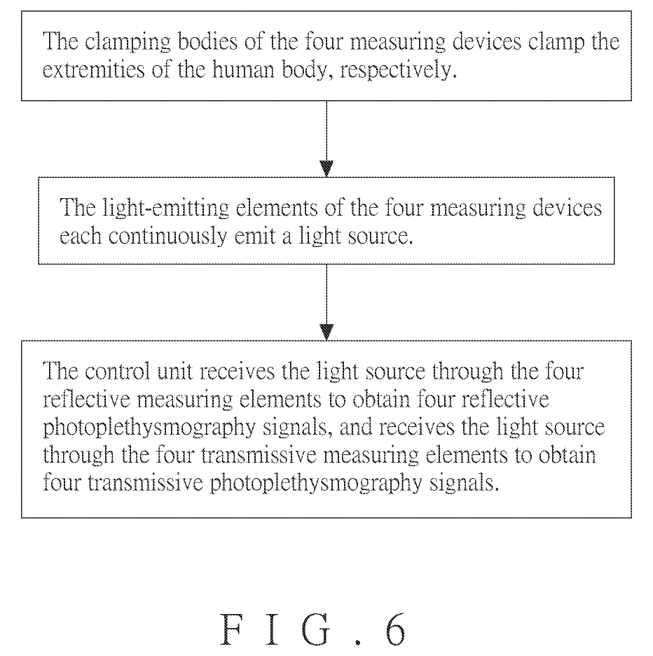

Referring to FIG. 1, FIG. 5 and FIG. 6, because there is a temperature difference between the core temperature of the human body and the skin temperature of the extremities, the temperature difference has no fixed value and depends on environmental conditions and physiological conditions. Under normal room temperature conditions, the temperature difference may be about 2° C to 5° C; under cold conditions, due to the significant reduction in peripheral blood flow in the limbs, the temperature difference may reach more than 10°C. When the temperature of the extremities is below 35° C, the measured photoplethysmography (PPG) is not accurate. The control unit 2 of the present invention uses the temperature sensors 16 of the four measuring devices 1 to detect the four extremities of the human body and obtain four extremity temperatures. When one of the extremity temperatures falls within a predetermined range, the control unit 2 activates the heating unit to perform heating. Specifically, the predetermined range is between 31 degrees Celsius and 35 degrees Celsius. When the detected extremity temperature is between 31° C and 35° C, the control unit 2 automatically activates the heating unit to perform heating, restoring the temperature of the extremities of the human body to normal, thereby obtaining a reflective photoplethysmography signal (rPPG) and a transmissive photoplethysmography signal (tPPG). For example, when the detected extremity temperature exceeds 38 degrees Celsius, the control unit 2 determines that the extremity temperature is abnormal and does not measure it. The QRS complex of the electrocardiogram represents the ventricular depolarization that initiates systemic blood ejection from the heart. Therefore, regardless of whether the electrocardiogram electrodes are placed on the chest or on the upper limbs, the detected R-wave peak still corresponds to the actual timing of the cardiac contraction event. Consequently, the R-wave obtained from the upper limb electrodes can serve as a reliable time reference for calculating pulse transit time (PTT) to each extremity.

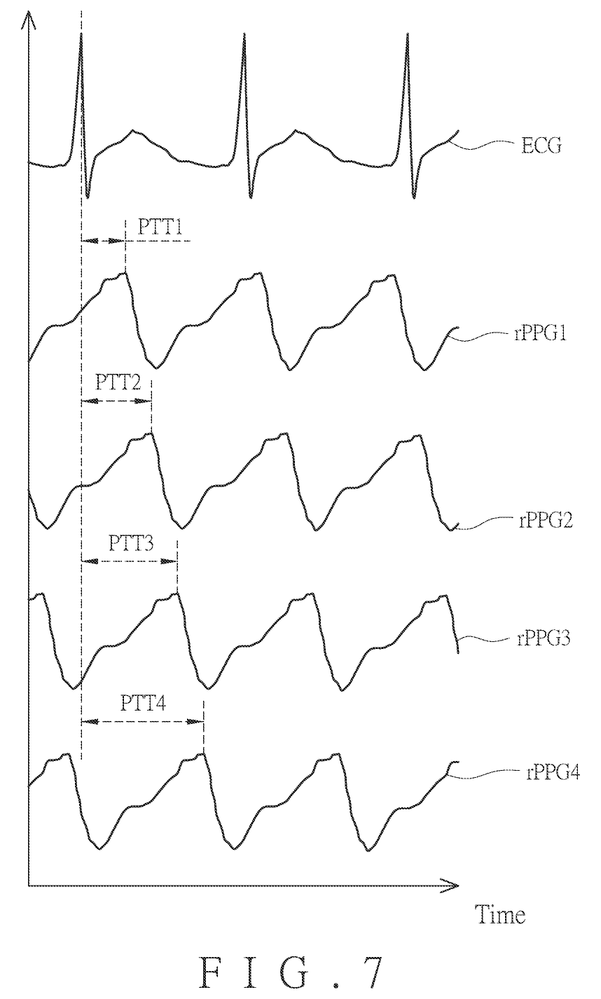

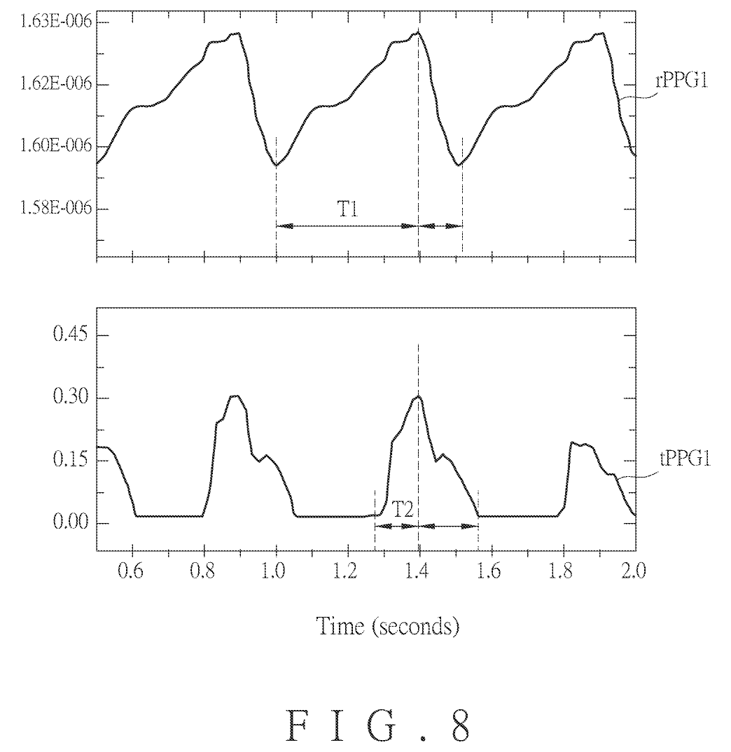

Referring to FIG. 1 and FIG. 7, the control unit 2 obtains an electrocardiogram signal ECG through the two electrocardiogram measuring electrodes 12 of the measuring devices 1 attached to the upper limbs. Referring to FIG. 1, FIG. 7 and FIG. 8, the light-emitting elements 13 of the four measuring devices 1 each continuously emit a light source. The control unit 2 receives the light source through the four reflective measuring elements 14 to obtain four reflective photoplethysmography signals, defined as a first reflective photoplethysmography signal rPPG1, a second reflective photoplethysmography signal rPPG2, a third reflective photoplethysmography signal rPPG3 and a fourth reflective photoplethysmography signal rPPG4. Besides, the control unit 2 receives the light source through the four transmissive measuring elements 15 to obtain four transmissive photoplethysmography signals, defined as a first transmissive photoplethysmography signal tPPG1, a second transmissive photoplethysmography signal, a third transmissive photoplethysmography signal and a fourth transmissive photoplethysmography signal. The second transmissive photoplethysmography signal, the third transmissive photoplethysmography signal and the fourth transmissive photoplethysmography signal are not shown in the figures. FIG. 8 only illustrates the difference between the first reflective photoplethysmography signal rPPG1 and the first transmissive photoplethysmography signal tPPG1.

In this embodiment, the reflective photoplethysmography signal and the transmissive photoplethysmography signal are obtained simultaneously in a synchronous measurement manner. When the light-emitting element 13 emits the light source, the reflective measuring element 14 and the transmissive measuring element 15 receive the light source simultaneously to obtain the reflective photoplethysmography signal and the transmissive photoplethysmography signal simultaneously. Besides, in the present invention, the reflective photoplethysmography signal and the transmissive photoplethysmography signal may be obtained in a staged measurement manner. For example, after the light-emitting element 13 emits the light source, the reflective measuring element 14 first measures the reflective photoplethysmography signal from 0 to 60 seconds, and then the transmissive measuring element 15 measures the transmissive photoplethysmography signal from 61 to 120 seconds. The staged measurement manner is a feasible implementation of the present invention.

Since the peak times of the reflective photoplethysmography signal and the transmissive photoplethysmography signal are the same, the following description is based on the reflective photoplethysmography signal.

The control unit 2 generates a first pulse transit time difference PTT1 based on the peak time difference between the electrocardiogram signal ECG and the first reflective photoplethysmography signal rPPG1, a second pulse transit time difference PTT2 based on the peak time difference between the electrocardiogram signal ECG and the second reflective photoplethysmography signal rPPG2, a third pulse transit time difference PTT3 based on the peak time difference between the electrocardiogram signal ECG and the third reflective photoplethysmography signal rPPG3, and a fourth first pulse transit time difference PTT4 based on the peak time difference between the electrocardiogram signal ECG and the fourth reflective photoplethysmography signal rPPG4.

By comparing the first pulse transit time difference (PTT1), the second pulse transit time difference (PTT2), the third pulse transit time difference (PTT3) and the fourth pulse transit time difference (PTT4), together with the ankle-brachial index (ABI) of the existing technique, it is possible to evaluate the vascular obstruction condition from the human heart to the extremities of the human body. Specifically, the time difference between the ventricular contraction of the heartbeat and the moment when blood is delivered to pass through the peripheral vessels of the extremities of the human body is defined as the peak time difference. When the peak time difference is large, it means that the blood takes a long time to flow, which may be caused by an obstruction in the vascular pathway. Therefore, the vascular obstruction condition of the extremities of the human body can be evaluated by comparing the first pulse transit time difference PTT1, the second pulse transit time difference PTT2, the third pulse transit time difference PTT3 and the fourth pulse transit time difference PTT4.

FIG. 7 and FIG. 8 show the first reflective photoplethysmography signal rPPG1 and the first transmissive photoplethysmography signal tPPG1; the second reflective photoplethysmography signal rPPG2 and the second transmissive photoplethysmography signal; the third reflective photoplethysmography signal rPPG3 and the third transmissive photoplethysmography signal; and the fourth reflective photoplethysmography signal rPPG4 and the fourth transmissive photoplethysmography signal measured at the extremities of the human body, respectively. In this embodiment, the first reflective photoplethysmography signal rPPG1 and the first transmissive photoplethysmography signal tPPG1 measured at one extremity are taken as an example. The second transmissive photoplethysmography signal, the third transmissive photoplethysmography signal and the fourth transmissive photoplethysmography signal are analyzed in the same manner, so they are not shown in the figures.

The first reflective photoplethysmography signal rPPG1 has a reflective systolic time T1. The first transmissive photoplethysmography signal tPPG1 has a transmissive systolic time T2. By analyzing the difference between the reflective systolic time T1 and the transmissive systolic time T2, together with the waveform analysis of the first reflective photoplethysmography signal rPPG1 and the first transmissive photoplethysmography signal tPPG1, the local vascular stiffness and elasticity conditions of one of the extremities can be further evaluated. Specifically, the reflective photoplethysmography signal measured by the diffusion of the light source in the vascular tissue. The reflective photoplethysmography signal reflects the calcification and lesions of microvessels in the tissue, thereby evaluating the conditions of microvessels. The transmissive photoplethysmography signal is a signal measured by the light source passing through the vascular tissue. The transmissive photoplethysmography signal reflects the stiffness and elasticity of vascular walls in the tissue, thereby evaluating the overall conditions of blood vessels. By analyzing both the reflective photoplethysmography signal and the transmissive photoplethysmography signal, the elasticity of blood vessels at the major blood vessels and microcirculation can be evaluated more comprehensively. The reflective photoplethysmography signal (rPPG) is primarily generated by light that penetrates only shallow layers of the tissue and is then scattered back to the detector. Because of the shorter optical path, rPPG is more sensitive to superficial microvascular circulation. In contrast, the transmissive photoplethysmography signal (tPPG) is generated by light passing entirely through the tissue before reaching the detector. This longer optical path causes tPPG to be influenced more by deeper vascular structures, including larger blood vessels. Therefore, by comparing the waveform characteristics and timing differences between rPPG and tPPG, the apparatus can evaluate vascular elasticity at both superficial microcirculation and deeper vessels, providing a more comprehensive assessment of peripheral circulation.

Furthermore, by analyzing the waveforms of the first reflective photoplethysmography signal rPPG1 or the first transmissive photoplethysmography signal tPPG1, characteristic parameters such as the number of arterial notches, the delay time of the second wave, and the slope of the waveform’s descent are utilized to calculate a vascular age index, thereby evaluating the vascular elasticity more comprehensively.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. An apparatus for monitoring peripheral circulation in human limbs by integrating temperature feedback with composite optical measurement techniques, comprising:
four measuring devices, each including a clamping body having clamping force, the clamping body being configured for clamping extremities of a human body, the clamping body having a clamping bottom surface and an opposing clamping top surface, two electrocardiogram measuring electrodes being disposed on the clamping bottom surface and the clamping top surface, a light-emitting element, a reflective measuring element and a temperature sensor being disposed on the clamping bottom surface, a transmissive measuring element being disposed on the clamping top surface, the reflective measuring element being adjacent to the light-emitting element, the transmissive measuring element facing the light-emitting element;
a heating unit, selectively increasing a human body temperature or an ambient temperature;
a control unit, connected to the heating unit and the two electrocardiogram measuring electrodes, the light-emitting element, the reflective measuring element, the transmissive measuring element and the temperature sensor of each of the four measuring devices;
wherein when in use, the four measuring devices clamp the extremities of the human body, respectively; the control unit detects the extremities of the human body through the temperature sensors of the four measuring devices to obtain an extremity temperature, when the extremity temperature of any one of the extremities of the human body falls within a predetermined range, the control unit activates the heating unit to perform heating; the control unit obtains an electrocardiogram signal through the electrocardiogram measuring electrodes of the measuring devices attached to the upper extremities; the light-emitting elements of the four measuring devices each continuously emit a light source, the control unit receives the light source through the four reflective measuring elements to obtain four reflective photoplethysmography signals and receives the light source through the four transmissive measuring elements to obtain four transmissive photoplethysmography signals; the control unit evaluates a vascular blockage condition from the heart to the extremities of the human body through peak time differences between the electrocardiogram signal and the four reflective photoplethysmography signals or through peak time differences between the electrocardiogram signal and the four transmissive photoplethysmography signals; and the control unit evaluates local blood vessel stiffness and elasticity conditions of each of the extremities of the human body through a difference between a corresponding one of the four reflective photoplethysmography signals and a corresponding one of the four transmissive photoplethysmography signals of each of the extremities of the human body.

2. The apparatus as claimed in claim 1, wherein the predetermined range is between 31 degrees Celsius and 35 degrees Celsius.

3. The apparatus as claimed in claim 1, wherein the electrocardiogram measuring electrode on the clamping top surface surrounds the transmissive measuring element, and the electrocardiogram measuring electrode on the clamping bottom surface surrounds the reflective measuring element and the light-emitting element.

4. The apparatus as claimed in claim 1, wherein the reflective photoplethysmography signal has a reflective systolic time, the transmissive photoplethysmography signal has a transmissive systolic time, and the local blood vessel stiffness and elasticity conditions of each of the extremities of the human body are evaluated through a difference between the reflective systolic time and the transmissive systolic time.

5. The apparatus as claimed in claim 1, wherein the heating unit has four heaters each mounted in the clamping body, and the heaters selectively heat the extremities of the human body directly for increasing the human body temperature.

6. The apparatus as claimed in claim 1, wherein the heating unit is an electric heater that is arranged together with the human body in an environmental space, so that the heating unit selectively heats the environmental space for increasing the ambient temperature.

7. A method for monitoring peripheral circulation in human limbs by integrating temperature feedback with composite optical measurement techniques, comprising the following steps of:
providing four measuring devices to clamp extremities of a human body and a heating unit, wherein the measuring devices each include a temperature sensor, two electrocardiogram measuring electrodes, a light-emitting element, a reflective measuring element and a transmissive measuring element that are connected to a control unit, and the heating unit is connected to the control unit;
detecting the extremities of the human body through the temperature sensors of the four measuring devices to obtain an extremity temperature, wherein when the extremity temperature of any one of the extremities of the human body falls within a predetermined range, the control unit activates the heating unit to perform heating;
obtaining an electrocardiogram signal through the electrocardiogram measuring electrodes of the measuring devices attached to the upper extremities;
emitting a light source through each of the light-emitting elements of the four measuring devices, wherein the four reflective measuring elements receive the light source to obtain four reflective photoplethysmography signals, and the four transmissive measuring elements receive the light source to obtain four transmissive photoplethysmography signals;
evaluating a vascular blockage condition from the heart to the extremities of the human body through peak time differences between the electrocardiogram signal and the four reflective photoplethysmography signals, or through peak time differences between the electrocardiogram signal and the four transmissive photoplethysmography signals; and
evaluating local blood vessel stiffness and elasticity conditions of each of the extremities of the human body through a difference between a corresponding one of the four reflective photoplethysmography signals and a corresponding one of the four transmissive photoplethysmography signals of each of the extremities of the human body.

8. The method as claimed in claim 7, wherein the predetermined range is between 31 degrees Celsius and 35 degrees Celsius.

9. The method as claimed in claim 7, wherein the reflective photoplethysmography signal has a reflective systolic time, the transmissive photoplethysmography signal has a transmissive systolic time, and the local blood vessel stiffness and elasticity conditions of each of the extremities of the human body are evaluated by a difference between the reflective systolic time and the transmissive systolic time.
